(19) United States
(12) Patent Application Publication
(10) Pub. No.: US 2024/0197588 A1
(43) Pub. Date: Jun. 20, 2024

(54) ANTIMICROBIAL COMPOSITION

(71) Applicant: Conopco, Inc., d/b/a UNILEVER, Englewood Cliffs, NJ (US)

(72) Inventors: Prem CHANDAR, Closter, NJ (US); Gabriella Satchi Olivia FREY, Milford, CT (US); Sandeep GUPTA, Mumbai (IN); Lei HUANG, Trumbull, CT (US); Teanoosh MOADDEL, Watertown, CT (US); Rohini Sukumaran NAIR, Mumbai (IN); Bharath PALANISAMY, Bangalore (IN); Anat SHILOACH, Trumbull, CT (US)

(73) Assignee: Conopco, Inc., d/b/a UNILEVER, Englewood Cliffs, NJ (US)

(21) Appl. No.: 18/286,774

(22) PCT Filed: Apr. 20, 2022
(86) PCT No.: PCT/EP2022/060374

§ 371 Date: Oct. 13, 2023

(30) Foreign Application Priority Data
Apr. 21, 2021 (IN) 202121018488
Jun. 10, 2021 (EP) 21178818.7

(51) Int. Cl. A61K 8/34; A61K 8/36; A61K 8/67; A61K 8/81; A61Q 17/00

(52) U.S. Cl. CPC A61K 8/34; A61K 8/361; A61K 8/675; A61K 8/678; A61K 8/817; A61Q 17/005; A61K 2800/262; A61K 2800/5426

(57) ABSTRACT

An antimicrobial composition comprises water; alcohol; a cationic polymer; and a detackifying agent.

### FIELD OF THE INVENTION

Disclosed herein is an antimicrobial composition, specifically, a transparent antimicrobial composition. The antimicrobial composition includes water, alcohol, a cationic polymer, and a detackifying agent.

### BACKGROUND OF THE INVENTION

Consumers are increasingly concerned with microbial and viral contamination. As such, hygiene continues to be one of the most important attributes desired among consumers. Consumers desire products that will provide the required hygienic properties. Consumers throughout the world use different types of antimicrobial compositions for disinfecting various surfaces including hard surfaces such as countertops, floors, and furniture; soft and porous surfaces such as clothes, carpets, and upholstery; and personal surfaces such as skin and hair. Many microorganisms like bacteria and viruses are found on such surfaces. It is desirable to keep these surfaces free of germs to minimize the risk of becoming ill.

Additionally, respiratory infections are considered to be one of the most prevalent cause of disease, worldwide. Scientific studies have identified respiratory syncytial virus (RSV), as the most common viral cause of death due to such infection; other prominent viruses being human metapneumovirus, parainfluenza viruses, influenza viruses A and B, adenoviruses, and of recent origin, coronavirus.

Other than viral infections, bacterial infections also account for a large percentage of morbidity and mortality in the world. Bacteria found on the skin can be divided into two groups: resident bacteria and transient bacteria. Resident bacteria are Gram positive bacteria which are established as permanent microcolonies on the surface and outermost layers of the skin and play an important, helpful role in preventing the colonization of other, more harmful bacteria and fungi. Transient bacteria are bacteria which are not part of the normal resident flora of the skin but can be deposited when airborne contaminated material lands on the skin or when contaminated material is brought into physical contact with the skin. Transient bacteria are also typically divided into Gram positive and Gram-negative subclasses. Gram positive bacteria include pathogens such as Staphylococcus aureus, Streptococcus pyogenes and Clostridium botulinum. Gram negative bacteria include pathogens such as Salmonella, Escherichia coli, Klebsiella, Haemophilus, Pseudomonas aeruginosa, Proteus and Shigella dysenteries. Gram negative bacteria are distinguished from Gram positive by an additional protective cell membrane which generally results in the Gram-negative bacteria being less susceptible to topical antimicrobial actives.

Research has shown that virus and bacteria as discussed above, can transmit through touching infected hands and even through inanimate surfaces including mobile phones. As a consequence, hand hygiene is a commonly recommended method of killing such germs and thereby reducing the risk of not only respiratory infection but also gastrointestinal diseases. Hand hygiene includes the use of soap and running water to wash the hands which clears dirt and kills germs including bacteria and virus. Alternatively, alcohol-based hand sanitizers which contain very little water may be rubbed on the hands as another effective means of removing virus and bacteria from hands. While such methods are good at instantaneously killing germs, they do not provide long lasting post wash or application protection from germs which may invade the surface for up to several hours after the surface has been cleaned. Long lasting protection from germs would require an additional step of applying a leave-on composition on to the surface.

As such, there is continually a need to develop an antimicrobial composition comprising an antimicrobial active that not only kills germs but also deposits actives in sufficient quantities on the skin to ensure effective germ kill for many hours after washing.

### SUMMARY OF THE INVENTION

Disclosed in various aspects herein are antimicrobial compositions.

An antimicrobial composition comprises water; alcohol; a cationic polymer; and a detackifying agent.

These and other features and characteristics are more particularly described below.

### DETAILED DESCRIPTION OF THE INVENTION

Disclosed herein is an antimicrobial composition with desirable (e.g., non-tacky) sensory properties and long lasting antimicrobial efficacy. The antimicrobial composition contains water, alcohol, a cationic polymer, and a detackifying agent. The cationic polymer assists in providing the antimicrobial composition with long lasting antimicrobial efficacy, but also can impart an unpleasant or sticky feeling to the user after application of the antimicrobial composition. A detackifying agent can help mitigate the stickiness associated with the inclusion of the cationic polymer, thus providing an antimicrobial composition with long lasting germ kill and a non-tacky feeling after application. The antimicrobial composition can be transparent or translucent, preferably the antimicrobial composition is transparent. For the transparent antimicrobial compositions, it was expected that the inclusion of a dectackifying agent would affect the transparency negatively. However, it was unexpectedly found that the inclusion of the detackifying agents disclosed herein did not negatively affect the transparency of the antimicrobial composition.

Sanitizing or antimicrobial composition as used herein refers to a composition that is capable of killing bacteria or inhibiting the growth of bacteria as well as fungi, parasites, algae, dust-mites, and certain viruses. Antimicrobial composition as used herein refers to a composition that continuously inhibits the growth of microbes on surfaces for extended periods of time. The antimicrobial compositions can be substantially emulsifier free. Substantially emulsifier free means that the composition contains less than 2% by weight emulsifier, preferably, less than 1% by weight emulsifier, more preferably less than 0.5% by weight emulsifier, even more preferably, less than 0.1% by weight emulsifier, still more preferably, 0% by weight emulsifier.

The antimicrobial composition contains a cationic polymer. The cationic polymer provides long term germ killing efficacy of the antimicrobial composition. The cationic polymer can generally be any cationic polymer but is preferably selected from polyquaternium-6 (poly diallyl dimethyl ammonium chloride (PDADMAC)), poly N-[3-(dimethylamino)propyl]methacrylamide (PDMAPMA), poly[2 (Dimethylamino)ethyl methacrylate] (PDMAEMA), polyethylene imine (PEI), chitosan, or a combination thereof. Preferably, the cationic polymer is polyquaternium-6.

The cationic polymers used in the compositions disclosed herein are preferably homo polymer/copolymers having at least one nitrogen atom as part of its repeating unit. The nitrogen is present so as to include a % nitrogen of greater than or equal to 5% by weight of the cationic polymer. Preferably, nitrogen is present in an amount of 5 to 45% by weight of the cationic polymer, more preferably, 6 to 40% by weight, and most preferably, 6 to 35% by weight.

Weight percent of nitrogen is calculated using the following equation:

Weight % of N=(Weight of N in repeating unit/total molecular weight of repeating unit)*100

Hence, weight percent of nitrogen does not vary with degree of polymerisation/molecular weight of polymer.

The weight percent nitrogen of cationic polymers used herein are:
PQ-6 (8.7%), PQ-37 (7.2%) and PQ-16 (16%). Cationic polymers outside the range disclosed include PQ-10 (1.5 to 2.2% N) and PQ-67 (1.3% N).

The preferred PDADMAC molecular weight (weight average molecular weight) for use in the antimicrobial compositions disclosed herein is 2,00,000 to 20,00,000, preferably 4,00,000 to 6,00,000. It is a cationic polymer with a viscosity of 10,000 to 20,000 mPa·s.

The polymer is available under the trade name of Merquat-100 (INCI: Polyquaternium-6) from Lubrizol, Inc.

The cationic polymer can be present in an amount of 0.1 to 5.0% by weight of the overall antimicrobial composition including all values and ranges subsumed therein, preferably, 0.25 to 4.0% by weight, more preferably, 0.5 to 2.0% by weight.

The antimicrobial composition comprises water in an amount of 8 to 40% by weight of the overall antimicrobial composition including all values and ranges subsumed therein, preferably 10 to 35% by weight, more preferably 12 to 30% by weight.

Alcohol can be used as the antimicrobial agent in the composition. When alcohol is used as the antimicrobial agent, the antimicrobial composition comprises alcohol in an amount of 30 to 80% by weight, preferably 40 to 78% by weight, more preferably 55 to 75% by weight, even more preferably 60 to 70% by weight, including all values and ranges subsumed therein. The alcohol can comprise a variety of alcohols including, but not limited to, ethanol, propanol (n-propanol), isopropyl alcohol (isopropanol), butanol, or a combination thereof. The level of alcohol present in the antimicrobial composition can be adjusted based upon the purity percentage of the alcohol. For example, if using ethanol with a purity of 92%, ethanol can be used in an amount of 70 to 80% by weight, for example, 75% by weight. Optionally, the alcohol can comprise chloroxylenol. A preferred alcohol can be denatured SD-40 ethanol.

The antimicrobial composition can optionally comprise one or more additional antimicrobial agents. The optional, additional antimicrobial agent can be selected from terpenes, essential oils, or cationic oils having a solubility in water of less than 2000 parts per million (ppm) at 25° C. Examples of aromatic essential oils that can be used in the antimicrobial compositions disclosed herein include amyl salicylate, carvacrol, cymene, e.g., p-cymene, dihydroeugenol, eugenol, hexyl eugenol, hexyl salicylate, isoeugenol, methyl eugenol, methyl isoeugenol, methyl salicylate, tert butyl cresol, thymol, and vanillin. Examples of non-aromatic essential oils of terpenoid compounds include cedrane, cineole, citral (including geranial and neral), citronellal, nitronelol, eucalyptol (i.e., 1,8 cineole) paradihydrolinalool, dihydromyrcenol (DH myrcenol), farnesol, geraniol, hexyl cinnamaldehyde, hydroxycitronallol, hydroxycitronellal, isocitral, limonene, preferably d-limonene, linallol, longifolene, menthol, nerol, nerolidiol, pinene, e.g., α-pinene, phellendrene, terpinine, e.g., α-terpinene and γ-terpinene, terpineol, e.g., γ-terpineol and terpin-4-ol, and tetrahydromyrcenol (THM).

Preferred cationic oils include quaternary ammonium cationic vegetable oil and charged aminopolydimethylsilane having the formula (CH₃)₃—Si[Si(CH₃)₂—O]—[Si(CH₃)—((CH₂)₃—NH—(CH₂)₂—NH₂)—O]₂—Si—(CH₃)₃. It is preferred that the solubility of these additional antimicrobial agents be less than 2000 ppm at 25° C. For example, the additional antimicrobial agent can be terpineol, thymol, eugenol, borneol, limonene, or a combination thereof. A preferred additional antimicrobial agent can be terpineol, thymol, or eugenol, or a combination thereof.

Other types of optional additional antimicrobial agents include silver compounds. The silver compound can comprise a silver ion. For example, the silver ion can be selected from silver nitrate, silver acetate, silver oxide, silver sulfate, or a combination thereof. Preferably, the silver compound is silver nitrate.

Stated more specifically, the silver compounds optionally employed in the compositions are one or more water-soluble silver (I) compounds having a silver ion solubility of at least 1.0×10−4 mol/L (in water at 25° C.). Silver ion solubility, as referred to herein, is a value derived from a solubility product (Ksp) in water at 25° C., a well-known parameter that is reported in numerous sources. More particularly, silver ion solubility [Ag+], a value given in mol/L, may be calculated using the formula:

$$[ Ag + ] = {( Ksp \cdot x )}^{( 1 / ( x + 1 ) )} ,$$

wherein Ksp is the solubility product of the compound of interest in water at 25° C., and x represents the number of moles of silver ion per mole of compound. It has been found that silver (I) compounds having a silver ion solubility of at least 1×10⁻⁴ mol/L are desirable for use herein.

Among the silver compounds desirable for use herein are silver oxide, silver nitrate, silver acetate, silver sulfate, silver benzoate, silver salicylate, silver carbonate, silver citrate and silver phosphate, or a combination thereof, preferably wherein the silver compound is silver nitrate, silver acetate, silver oxide, silver sulfate, or a combination thereof.

When present, the additional antimicrobial agent can be included in an amount of 0.05 to 2% by weight of the overall antimicrobial composition including all values and ranges subsumed therein, for example, 0.1 to 2% by weight.

A detackifying agent can be used in the antimicrobial composition to assist in mitigating any tacky/sticky feeling left on the skin after application of the antimicrobial composition comprising cationic polymer. The antimicrobial composition comprises a detackifying agent in an amount of 0.01 to 15% by weight of the overall antimicrobial composition, including all values and ranges subsumed therein, preferably, 0.01 to 10% by weight of the overall antimicrobial composition including all values and ranges subsumed therein, more preferably, 0.5 to 10% by weight of the overall antimicrobial composition including all values and ranges subsumed therein, even more preferably, 0.5 to 5% by weight of the overall antimicrobial composition including all values and ranges subsumed therein.

Detackifying agents desirable for use in the antimicrobial composition disclosed herein must meet the following criteria: the detackifying agent must be liquid, the detackifying agent must be soluble in at least a 75/25 or higher ratio of ethanol to water (EtOH/Water), which is to be reflective of solubility in the final composition resulting in a clear (i.e., transparent) final antimicrobial composition, and the detackifying agent must have no solvency for water.

The detackifying agent can comprise a soluble detackifying agent. Solubility assists in ensuring a transparent antimicrobial composition is produced. The soluble detackifying agent can comprise dicarboxylic acid esters, lactate esters (e.g., C12/14/16 lactate ester), (poly)propylene glycol fatty ethers, or a combination thereof.

The detackifying agent can also comprise mixtures of a soluble detackifying agent and an insoluble detackifying agent, provided that the combination of soluble and insoluble agents is soluble in the end use antimicrobial composition. For example, the soluble detackifying agent can comprise diisopropyl adipate, diisopropyl sebacate, diethyl hexyl malate, lauryl lactate, myristyl lactate, cetyl lactate, isostearyl alcohol, PPG15 stearyl ether, or a combination thereof.

Further combinations of the aforementioned detackifying agents with insoluble oils such as triglycerides (e.g., capric/caprylic triglyceride), soybean oil, or fatty esters (e.g., isopropyl myristate or isopropyl palmitate) are also in scope of the disclosed antimicrobial compositions provided that the oils are soluble and form a transparent end use antimicrobial composition. The latter is not meant to be limiting to the choice of insoluble detackifying agents that can be used in the compositions disclosed herein. That is, other insoluble oils can be used provided that when combined with the selected detackifying agents of the present compositions, the insoluble agents become soluble when mixed into with the end use antimicrobial composition. Any combination of detackifying agents used in the antimicrobial compositions shall not affect the transparency of the antimicrobial composition.

The antimicrobial composition can be in any form. For example, the antimicrobial composition can be in the form of a liquid, a gel, or a spray.

When the antimicrobial composition is in the form of a liquid or a gel, a structurant can be present in the composition. The structurant can comprise a nonionic structurant, a cationic structurant, or a combination thereof. Without wishing to be bound by theory, it is believed that an anionic structurant will react with the cationic polymer and precipitate. Thus, it is not desirable to use an anionic structurant in the disclosed antimicrobial compositions.

Structurants can also be used to adjust antimicrobial composition viscosity and to assist in forming the composition (e.g., giving it structure) in a non-spray form. When present, structurants can be used in an amount of 0.01% by weight to 3% by weight of the overall antimicrobial composition including all values and ranges subsumed therein, for example, 0.1% by weight to 2.5% by weight, for example, 0.15% by weight to 2.0% by weight, for example, 0.20% by weight to 1.5% by weight of the overall antimicrobial composition. The amount of structurant to be used can be adjusted based on the desired end use viscosity.

The structurant can comprise cellulosics which can include hydroxypropyl cellulose (e.g., Klucel series from Ashland), hydroxypropyl methylcellulose (e.g., BENECEL™ E10M by Ashland), hydroxyethylcellulose (e.g. Natrosol series from Ashland), and cellulose (e.g. cellulose microfibrils, cellulose nanocrystals or microcrystalline cellulose).

Further nonionic structurants can also include maltodextrin, pentaerythrityl tetrastearate or a combination thereof.

Cationic structurants, including cationic polymeric structurants are also suitable for use. As to the cationic polymeric structurants, the same includes polymers classified as cationic guar gum derivatives, synthetic cationic polymers, and cationic starch derivatives. Those classified as partially cationically substituted quaternary ammonium salts are often desired for use.

Suitable polymers of this type conform to the structure given below:

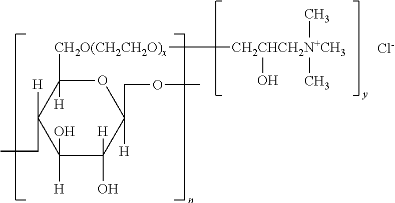

wherein, x is 0 to 3, preferably 0 to 1; and the ratio of y:n is 0.01 to 0.5 (i.e., n:y=100 to 2). It is desirable that cationic polymers with fully quaternized sugar units, i.e., y=n, are excluded.

The ratio of unquaternized to quaternized sugar units (n:y) is preferably 3 to 30, and more preferably, 4 to 25, most preferably 5 to 20.

The weight average molecular weight of polymers is in the range of 100 to 3,000,000 kiloDaltons (kDa), preferably, 500 to 1,000,000 kDa, more preferably, 10,000 to 500,000 kDa, and mixtures of polymers may be used. In an embodiment, the weight average molecular weight of the polymer is 150 to 100,000 kDa. In another embodiment, the weight average molecular weight of the polymer is 200 to 3,000 kDa. In still another embodiment, the weight average molecular weight of the polymer is 250 to 2,500 kDa.

Preferred cationic polymeric structurants for use are referred to as quaternary nitrogen-containing polysaccharides, more preferably, quaternary nitrogen-containing cellulose ethers, such as those described in U.S. Pat. Nos. 3,472,840; 3,962,418; 4,663,159, 5,407,919 and International Publication No. WO2005/000903 A1, the disclosures of which are incorporated herein by reference. Particularly preferred cationic polymeric structurants are quaternary nitrogen containing hydroxyethyl celluloses. Suitable examples of cationic polymeric structurants are salts of hydroxyethyl cellulose reacted with a trimethyl ammonium substituted epoxide, referred to in the industry by the Cosmetic, Toiletry, and Fragrance Association (CTFA) as polyquaternium-10 (PQ-10), the same being commercially available from Amerchol Corporation, a subsidiary of The Dow Chemical Company, as UCARE™ Polymer JR-125, UCARE™ Polymer JR-400, UCARE™ Polymer KF, UCARE™ Polymer JR-30M, UCARE™ Polymer LR-400, UCARE™ Polymer LR-30M, UCARE™ Polymer KG-30M, and UCARE™ Polymer LK. Other commercially available PQ-10 materials are KG30 or SENSOMER™ 10M from Lubrizol, Inc.

Examples of other preferred cationic polymeric structurants are referred to by CTFA polyquaternium-67. They are commercially available from Amerchol Corp. as the SoftCAT™ polymers like SoftCAT™ SL 5, SoftCAT™ SL 30, SoftCAT™ SL 60, SoftCAT™ SL 100, SoftCAT™ SK-L, SoftCAT™ SK-M, SoftCAT™ SK-MH, SoftCAT™ SK-H, SoftCAT™ SX-400X, SoftCAT™ SX-400H, SoftCAT™ SX-1300X and SoftCAT™ SX-1300H. Other examples of preferred cationic polymeric structurants are those referred to in the industry by the CTFA as polyquaternium-7 with the CAS Registry Number 026590-05-6, and those referred by the CTFA as polyquaternium-44. Still other cationic polymeric structurants include JAGUAR® C13S, JAGUAR® C14S, and JAGUAR® C17 made commercially available from Solvay. Even other types of cationic cellulose ethers include the polymeric quaternary ammonium salts of hydroxyethyl cellulose reacted with lauryl dimethyl ammonium-substituted epoxide referred to in the industry (CTFA) as polyquaternium-24. Polyquaternium-32, polyquaternium-37 polyquaternium-16, polyquaternium-45, polyquaternium-28, polyquaternium-53 can also be used. Any combination of the above mentioned cationic polymeric structurants can be used as a structurant in the disclosed antimicrobial compositions.

As to the percent substitution of nitrogen by weight (i.e., cationic substitution) within the cationic polymer, typically the percent nitrogen is 0.1 to 4%, and preferably, 0.3 to 3.5%, and most preferably, 1 to 2.8% by weight, based on total weight of the cationic polymer.

Surfactants of the cationic class which can be included in the antimicrobial compositions include cetyltrimethylammonium chloride (CTAC), cetyltrimethylammonium bromide (CTAB), dodecyltrimethylammonium chloride (DTAC), or a combination thereof.

The antimicrobial composition can additionally contain other ingredients in addition to those previously described herein including, but not limited to, skin benefit agents, fragrances, preservatives, surfactants, fixatives, opacifiers, chelators, structurants, humectants, dyes or colorants, or a combination thereof. For example, various colorants can optionally be used in the antimicrobial composition. When present, the colorants can be present in an amount of 0.00001 to 0.005% by weight of the overall antimicrobial composition including all values and ranges subsumed therein, for example, 0.0001 to 0.003% by weight, for example, 0.001% by weight.

For example, humectants can optionally be used in the antimicrobial composition to provide additional moisturization properties to the composition. Such humectants desirable for use in the antimicrobial composition can include water soluble polyols such as propylene glycol, dipropylene glycol, polypropylene glycol (e.g., PPG-9), polyethylene glycol, hydroxypropyl sorbitol, sorbitol, hexylene glycol, 1,3-butylene glycol, 1,4-butylene glycol, 1,2-octane diol, 1,2-hexane diol, isoprene glycol, 1,2,6-hexanetriol, ethoxylated glycerol, propoxylated glycerol, and combinations thereof. Most preferred are glycerin, butylene glycol, propylene glycol, polyethylene glycols, sorbitol, polyglycerol, isoprene glycol, hyaluronic acid, or a combination thereof. The humectant can be present in an amount of 1 to 15% by weight, preferably 2 to 10% by weight, more preferably 3 to 8% by weight of the antimicrobial composition, including any and all combinations subsumed therein.

The antimicrobial composition can optionally contain surfactants, for example, cationic surfactants. When used, the cationic surfactant is only limited in that it should be able to be used for topical application onto human skin. The cationic surfactant can comprise branched or straight chain alkyl trimonium compounds, alkanol trimonium compounds or a combination thereof. The alkanol trimonium compounds include lauroyl ethyltrimonium methosulfate, palmitoyl ethyltrimonium methosulfate, stearoyl ethyltrimonium methosulfate, carnitine, palmitoyl carnitine, a combination thereof or the like. The trimonium compound used can be an alkyl trimonium compound comprising cetrimonium chloride, cetrimonium bromide, mytrimonium chloride, mytrimonium bromide, behentrimonium methosulfate, cocotrimonium methosulfate, behentrimonium chloride, behentrimonium bromide, steartrimonium chloride, steartrimonium bromide, laurtrimonium chloride, laurtrimonium bromide, a combination thereof or the like. For the avoidance of doubt, when used, the cationic surfactant used in the antibacterial composition disclosed herein can consist essentially of or consist of any combination of the aforementioned surfactants.

As to the cationic surfactant comprising a dimonium compound, such a compound includes dialkyl dimonium compounds like distearyl dimonium chloride, didecyl dimonium chloride, dicoco dimonium chloride, a combination thereof or the like. Other dimonium compounds suitable for use include benzethonium chloride and/or benzalkonium chloride. The dimonium and trimonium compounds used herein are meant to include salts of the same, especially chlorides and bromides of the same.

The amount of cationic surfactant (i.e., cationic trimonium, dimonium) and/or polyquaternium material used in the composition is typically 0.007 to 5% by weight of the overall antimicrobial composition including all values and ranges subsumed therein, and preferably, from 0.01 to 3% by weight, and most preferably, from 0.05 to 2% by weight.

When both trimonium and dimonium surfactant are used they are often used in a weight ratio of 1:99 to 99:1, preferably, 30:70 to 70:30, and most preferably, 40:60 to 60:40.

The antimicrobial composition can also optionally include sunscreens and photostabilizers, provided that the type and amount of sunscreens and photostabilizers used do not affect the transparency and antimicrobial efficacy of the composition. The sunscreens and photostabilizers for use include such materials as octylmethoxycinnamate (OMC), ethylhexyl salicylate, phenylbenzimidazole sulfonic acid (Ensulizole), ethylhexyl p-methoxycinnamate, available as PARSOL® MCX, Avobenzene (butyl methoxydibenzoylmethane), available as PARSOL® 1789, benzophenone-3, also known as oxybenzone and benzophenone-4, also known as sulisobenzone. Still others can include bis-ethyl hexyloxyphenol methoxyphenol triazine, 2-ethylhexyl-2-cyano3,3-diphenyl-2-propanoic acid, drometrizole trisiloxane, 3,3,5-trimethyl cyclohexyl 2-hydroxybenzoate, 2-ethylhexyl-2-hydroxybenzoate or combination thereof. Inorganic sunscreen actives may be employed such as microfine titanium dioxide (preferably with a particle diameter of less than 150 nanometers (nm), and most preferably, less than 100 nm) and zinc oxide may be used, polyethylene and various other polymers are also suitable sunscreens. Other sunscreens suitable for use include p-aminobenzoic acid (PABA), octyldimethyl-PABA, 2-ethoxyethyl p-methoxy cinnamate, benzophenone-1, benzophenone-2, benzophenone-6, benzophenone-8, benzophenone-9, benzophenone-12, homomethyl salicylate, menthyl anthranilate, benzophenone-4, triethanolamine salicylate, terephthalylidene dicamphor sulfonic acid, bisoctriazole, bisethylhexyloxyphenol methoxyphenyl triazine, bisdisulizole disodium, diometriazole trisiloxane, octyltriazone, iscotrizinol, polysilicone-15, isopentenyl-4-methoxycinnamate, or a combination thereof. Octocrylene can also be used. Amounts of the sunscreen or photostabilizing agents when present can be 0.001 to 20% by weight of the overall antimicrobial composition including all values and ranges subsumed therein, preferably, 0.005 to 15% by weight, more preferably 0.01 to 0.2% by weight, even more preferably, 0.1% by weight.

Desirably the optional skin benefit agents used in the antimicrobial composition disclosed herein include niacinamide (vitamin B₃), tocopherol (Vitamin E), aloe vera, alpha-hydroxy acids and esters, beta-hydroxy acids and esters, hydroxyethyl urea, polyhydroxy acids and esters, creatine, hydroquinone, t-butyl hydroquinone, mulberry, hyaluronic acid and salts thereof (including, but not limited to, Na+ and K+ salts of the same), extract, liquorice extract, resorcinol derivatives, or a combination thereof. For example, the skin benefit agent can be sodium hyaluronate. Such benefit agents, including sodium hyaluronate can be present in an amount of 0.0001 to 10%, for example, 0.001 to 6.5%, for example, 0.01 to 3.5%, and for example, 0.01% by weight, based on total weight of the antimicrobial composition and including all values and ranges subsumed therein.

Further optional water-soluble skin benefit agents include acids, such as amino acids like arginine, valine or histidine. Other vitamins can be used such as vitamin B₂, picolinamide, panthenol (vitamin B₅), vitamin Br, vitamin C, a combination thereof or the like. Derivatives (generally meaning something that has developed or been obtained from something else), and especially, water soluble derivatives of such vitamins can also be employed. For instance, vitamin C derivatives such as ascorbyl tetraisopalmitate, magnesium ascorbyl phosphate, and ascorbyl glycoside may be used alone or in combination with each other. Niacinamide derivatives such as nicotinamide adenine dinucleotide (NADH) and nicotinamide adenine dinucleotide phosphate (NADPH) may be used alone or in combination with each other. Other skin benefit agents that can be used include 4-ethyl resorcinol, extracts like sage, aloe vera, green tea, sugar cane, citrus, grapeseed, thyme, chamomile, yarrow, cucumber, liquorice, rosemary extract or a combination thereof. Electrolytes such as NaCl, and/or KCl, and/or MgCl₂ may also be used. The total amount of optional water-soluble benefit agents (including mixtures) when present in the composition disclosed herein can be 0.0001 to 10%, preferably, 0.001 to 6.5%, and most preferably, 0.01 to 3.5% by weight, based on total weight of the antimicrobial composition, including all values and ranges subsumed therein.

It is also within the scope of the antimicrobial composition to optionally include oil soluble benefit agents. Illustrative examples of the types of oil soluble benefit agents that can optionally be used in the antimicrobial composition disclosed herein include components like stearic acid, vitamins like vitamin A, D, E, and K (and their oil soluble derivatives).

Other optional oil soluble benefit agents for use include resorcinols and resorcinol derivatives like 4-hexyl resorcinol, 4-phenylethyl resorcinol, 4-cyclopentyl resorcinol, 4-cyclohexyl resorcinol 4-isopropyl resorcinol or a combination thereof. Also, 5-substituted resorcinols like 4-cyclohexyl-5-methylbenzene-1,3-diol, 4-isopropyl-5-methylbenzene-1,3-diol, combination thereof or the like may be used. The 5-substituted resorcinols and their synthesis are described in commonly assigned U.S. Published Patent Application No. 2016/0000669A1.

Even other oil soluble benefit agents that can be used include omega-3 fatty acids, omega-6 fatty acids, climbazole, magnolol, honokiol, farnesol, ursolic acid, myristic acid, geranyl geraniol, oleyl betaine, cocoyl hydroxyethyl imidazoline, hexanoyl sphingosine, 12-hydroxystearic acid (12HSA), petroselinic acid, conjugated linoleic acid, stearic acid, palmitic acid, lauric acid, terpineol, thymol essential components, the dissolution auxiliary selected from limonene, pinene, camphene, cymene, citronellol, citronellal, geraniol, nerol, linalool, rhodinol, borneol, isoborneol, menthone, camphor, safrole, isosafrole, eugenol, isoeugenol, tea tree oil, eucalyptus oil, peppermint oil, neem oil, lemon grass oil, orange oil, bergamot oil, or a combination thereof.

Another optional oil soluble benefit agent that may be used is a retinoic acid precursor. The retinoic acid precursor can be retinol, retinal, retinyl ester, retinyl propionate, retinyl palmitate, retinyl acetate or a combination thereof. Retinyl propionate, retinyl palmitate and combinations thereof are typically preferred. Still another retinoic acid precursor for use is hydroxyanasatil retinoate made commercially available under the name RETEXTRA® as supplied by Molecular Design International. The same may be used in a combination with any of the oil soluble benefit agents described herein.

When an optional (i.e., 0.0 to 1.5% by weight) oil soluble benefit agent is used in the antimicrobial composition, it typically is present in an amount of 0.001 to 1.5% by weight of the overall antimicrobial composition including all values and ranges subsumed therein, and for example, 0.05 to 1.2% by weight, for example, 0.2 to 0.5% by weight of the total weight of the end use composition.

Film forming agents may be used in the antimicrobial compositions. While optional, such agents can aid with the composition adhering to the surface to which it is applied. Film forming agents include those having hydrophilic properties and they include materials comprising polyvinylpyrrolidone (PVP), acrylates, acrylamides, and copolymers thereof. Deposition agents like organosiloxanes and polyquaternium-7 (Merquat™ S Polymer from Lubrizol) may also be used. When used, such agents make up from 0.001 to 1% by weight of the antimicrobial composition including all values and ranges subsumed therein.

Other optional components that can be used in the composition are anti-mosquito agents like eucalyptus oil, lavender oil, citronella oil, N,N-diethyl-meta-toluamide (DEET), a combination thereof or the like. Even other ingredients which may be used include octopirox (piroctone), zinc pyrithione, chloroxylenol, triclosan, cetylpyridinium chloride as well as silver compounds including silver oxide, nitrate, sulfate, phosphate, carbonate, acetate, benzoate, a combination thereof or the like. If used, these other components typically make up from 0.001 to 1.6% by weight of the overall antimicrobial composition including all values and ranges subsumed therein, and preferably, from 0.01 to 1.2% by weight.

Optionally, preservatives can be used in the antimicrobial composition disclosed herein. When used, illustrative preservatives for use include sodium benzoate, iodopropynyl butyl carbamate, phenoxyethanol, hydroxyacetophenone, ethylhexylglycerine, methyl paraben, propyl paraben, imidazolidinyl urea, sodium dehydroacetate, dimethyl-dimethyl (DMDM) hydantoin and benzyl alcohol, or a combination thereof. Other preservatives suitable for use include sodium dehydroacetate, chlorophenesin and decylene glycol. Preservatives are preferably employed in amounts of 0.01% to 2.0% by weight of the total weight of antimicrobial composition, including all values and ranges subsumed therein. Also preferred is a preservative system with hydroxyacetophenone alone or in a mixture with other preservatives.

Fragrances, fixatives, opacifiers (like titanium dioxide or glycol distearate), chelating agents may optionally be included in the antimicrobial composition. Possible chelating agents include, but are not limited to, ethylyene diaminetetraacetic acid (EDTA), diethylenetriaminepentaacetic acid (DTPA), ethylene diamine disuccinic acid (EDDS), pentasodium diethylenetriaminepentaacetate, trisodium N-(hydroxyethyl)-ethylenediaminetracetate, an acid form of EDTA, sodium thiocynate, trisodium salt of methylglycinediacetic acid, tetrasodium glutamate diacetate and phytic acid, preferably wherein the chelating agent is ethylene diaminetetraacetic acid (EDTA), diethylenetriaminepentaacetic acid (DTPA), ethylene diamine disuccinic acid (EDDS), or a combination thereof. Each of these substances may be present in an amount of about 0.03 to about 3% by weight of the overall antimicrobial composition including all values and ranges subsumed therein, preferably, about 0.1 to about 2.6% by weight.

Another optional additive desirable for use includes hemp oil with 2.5 to 25% by weight cannabigerol and/or cannabidiol at from 0.5 to 10 percent by weight. When used, such oil makes up 0.0001 to 1.5% by weight of the overall antimicrobial composition including all values and ranges subsumed therein, and preferably, 0.01 to 1% by weight.

The antimicrobial composition can optionally contain an emulsifier. The emulsifier may be selected from the group consisting of those with a C₁₀-C₂₀ fatty alcohol or acid hydrophobe condensed with about 2 to about 100 moles of ethylene oxide or propylene oxide per mole of hydrophobe; C₂-C₁₀ alkyl phenols condensed with 2 to 20 moles of alkylene oxide; mono- and di-fatty acid esters of ethylene glycol; f; sorbitan, mono- and di-C₈-C₂₀ fatty acids; and polyoxyethylene sorbitan, or a combinations thereof. Alkyl polyglycosides and saccharide fatty amides (e.g., methyl gluconamides) can also be used as nonionic emulsifiers.

Preferred emulsifiers typically have an HLB (hydrophilic-lipophilic balance) of 7.5 to 20, and preferably, 8 to 18, and most preferably, 9 to 15, including all ranges subsumed therein. e.g., nonionic emulsifier can include polysorbate 20 (Tween 20), polyoxyethylene (20) sorbitan monooleate (Tween 80). When present, the emulsifier can be present in an amount of 0 to 1% by weight of the overall antimicrobial composition including all values and ranges subsumed therein, for example, 1% by weight.

As to packaging, the antimicrobial composition, can be packaged in pump bottle, a spray bottle, a squeeze bottle, or provided as an impregnating wetting agent on cotton swab, wipe, towelette, cosmetic substrate sheet (like those described in U.S. Pat. No. 6,294,182 B1) or the like. As the viscosity is increased with structurant, the antimicrobial composition gels and may be provided to consumers in a squeeze bottle as a gel composition. The spray bottle may also be metal, and the antimicrobial composition may be provided via conventional aerosol packaging technologies and including those which utilize air-in-bag discharging cannisters, mechanisms and actuators. It is also within the scope of the antimicrobial composition to include foaming agents (e.g., zwitterionic and/or amphoteric surfactants) so that the antimicrobial composition can be discharged as a foam.

The antimicrobial composition should be supplied with instructions to apply (e.g., squeeze or spray) the composition on to a surface, like skin, for bacteria kill and viral activity reduction. The antimicrobial composition can be provided for in biodegradable packaging and the packaging used is preferably refillable or reusable, biodegradable, and/or at least 50%, and preferably, at least 100% made from post-consumer recycled resin. The antimicrobial composition can be in the form of a liquid or a gel.

Skin, as used herein, is meant to include skin on the arms (including underarms), face, feet, neck, chest, hands, legs, buttocks and scalp (including hair). Sanitizing as used herein means a bacteria log kill of at least 2 and a viral log inactivation of at least 2 (both achieved), preferably a bacteria log kill of at least 3 and a viral log inactivation of at least in less than 3 minutes after topical application to a surface. Combination, as used herein means, total weight, e.g., of cetrimonium chloride plus benzalkonium chloride. Skin benefit agent means an ingredient suitable to improve a skin characteristic. Surface as used herein includes skin or the surface of an inanimate object such as a tabletop, computer monitor, doorknob, toilet seat, shopping cart handle or even a clothing garment. Surface is also meant to include the coat of an animal such as the fur on a dog and cat. As used herein, surface preferably means human skin, and especially, skin on the face and hands.

The antimicrobial composition can be a home care composition like a laundry spray composition suitable to spray clothing and upholstery requiring sanitizing. The home care composition can also be an antimicrobial kitchen or bathroom spray composition. Preferably, the antimicrobial composition is a topical composition to apply to skin for sanitizing by significantly reducing the amount of bacteria and viruses on the skin. The composition may optionally comprise skin benefit ingredients added thereto such as emollients, vitamins and/or derivatives thereof, resorcinols, retinoic acid precursors, colorants, moisturizers or humectants, fragrances, sunscreens, a combination thereof or the like as previously described herein. The skin benefit ingredients may be water or oil soluble.

The antimicrobial composition, therefore, is a hydroalcoholic based composition with a pH of 3.0 to 8.2, and the composition is water/alcohol continuous. Viscosity, as used herein, is taken either with a Brookfield viscometer using Spindle 4 at 10 rpm or with a Discovery HR-2 Rheometer using sand blasted plates having a 1000 micron gap and a first shear rate S_{A} of 0.4 s⁻¹ for a first viscosity V_{A} and a second shear rate S_{B} of 10 s⁻¹ for a second viscosity V_{B}, both at 25° C. and 20 second interval.

Several units are known to be used in the context of viscosity, but the ones used more often are centipoise (cP), Pascal seconds (Pa*s) and millipascal seconds (mPa*s) and these units are easily interconvertible with the help of publicly available resources like textbooks, encyclopedias and the internet. In still another embodiment, the composition is a non-therapeutic and non-medicinal composition which is a hydroalcoholic solution having a viscosity under 30,000 centipoises (cps) (30 Pa*s), preferably having a viscosity under 25,000 cps (25 Pa*s), preferably having a viscosity of 2,000-25,000 cps (2-25 Pa*s), more preferably having a viscosity of 2,000-10,000 cps (2-10 Pa*s).

Typically, the viscosity of the antimicrobial composition will be under 30,000 cps (30 Pa*s). Often the viscosity of the antimicrobial composition will be 1 to 25,000 cps (0.001 to 25 Pa*s), and preferably, 1500 to 25,000 cps (1.5 to 25 Pa*s), more preferably, 2,000 to 20,000 cps (2 to 20 Pa*s), and still more preferably, 2,000 to 10,000 cps (2 to 10 Pa*s), including all ranges subsumed therein.

The antimicrobial compositions can be made by any method of making an antimicrobial composition. In one embodiment, a method of making the antimicrobial composition can include the following: combining a detackifying agent and a humectant to form a first phase. Water is then combined with alcohol to form a second phase. The first and second phases are then combined to form a third phase. A neutralizer is added to the third phase, thereby forming the antimicrobial composition.

In the absence of explicitly stating otherwise, all ranges described herein are meant to include all ranges subsumed therein. As used herein, except where explicitly described, substantially free of means less than 10% by weight. Antimicrobial benefits mean at least a log kill of 2, preferably at least a log kill of 3, in under 3 minutes whereby antimicrobial assessment is measured via ASTM International standard method E2783-11 (Reapproved 2016) which sets forth the procedure for measuring antimicrobial activity for water miscible compounds using a time kill procedure. Viral (or virus) inactivation is determined by assessing the impact of microbiocides against viruses as set forth in ASTM International standard method 1052-20. The term comprising is meant to encompass the terms consisting essentially of and consisting of. For the avoidance of doubt, and for illustration, a composition comprising water, cationic surfactant and preservative is meant to include a composition consisting essentially of the same and a composition consisting of the same.

Except where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration or amount, any particular upper concentration can be associated with any particular lower concentration or amount as well as any subranges consumed therein. In that regard, it is noted that all ranges disclosed herein are inclusive of the endpoints, and the endpoints are independently combinable with each other (e.g., ranges of “up to 25% by weight, or, more specifically, 5% by weight to 20% by weight, in inclusive of the endpoints and all intermediate values of the ranges of 5% by weight to 25% by weight, etc.). “Combination is inclusive of blends, mixtures, alloys, reaction products, and the like. Furthermore, the terms “first”, “second”, and the like herein do not denote any order, quantity, or importance, but rather are used to distinguish one element from another. The terms “a” and “an” and “the” herein do not denote a limitation of quantity and are to be construed to cover both the singular and the plural, unless otherwise indicated herein or clearly contradicted by context. The suffix “(s)” as used herein is intended to include both the singular and the plural of the term it modifies, thereby including one or more of the term (e.g., the film(s) includes one or more films). Reference throughout the specification to “one embodiment”, “one aspect”, “another embodiment”, “another aspect”, “an embodiment”, “an aspect” and so forth means that a particular element (e.g., feature, structure, and/or characteristic) described in connection with the embodiment or aspect is included in at least one embodiment or aspect described herein and may or may not be present in other embodiments or aspects. In addition, it is to be understood that the described elements may be combined in any suitable manner in the various embodiments or aspects.

All cited patents, patent applications, and other references are incorporated herein by reference in their entirety. However, if a term in the present application contradicts or conflicts with a term in the incorporated reference, the term from the present application takes precedence over the conflicting term from the incorporated reference. While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

For the avoidance of doubt the word “comprising” is intended to mean “including” but not necessarily “consisting of” or “composed of.” In other words, the listed steps, options, or alternatives need not be exhaustive.

The disclosure of the invention as found herein is to be considered to cover all aspects as found in the claims as being multiply dependent upon each other irrespective of the fact that claims may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated. The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

### EXAMPLES

The following examples are merely illustrative of the antimicrobial compositions disclosed herein and are not intended to limit the scope hereof.

### Example 1

Samples 1 to 9 were examples demonstrating compositions of the antimicrobial compositions disclosed herein and Comparative Examples 1 to 11 (CE1 to CE11) were examples demonstrating compositions outside those disclosed herein. All viscosities were measured using a Brookfield Spindle 4 at 10 rpm.

The samples were made at room temperature, atmospheric pressure, and standard shear by dispersing a cationic polymer in water to form phase A. Alcohol formed phase B. Then any skin benefit agents, humectants, detackifying agents, and structurants (when included) were combined to form phase C.

First, phases A and B were combined and mixed with an overhead pneumatic mixer until a clear mixture was formed at which point Phase C was added. A neutralizer was then added as Phase D and a fragrance added in Phase E, thereby forming the antimicrobial composition. BENECEL™ E10M is a nonionic structurant, specifically hydroxypropyl methylcellulose. KLUCEL™ is a nonionic structurant, specifically hydroxypropylcelluslose. PQ-10 is a cationic structurant, specifically polyquaternium-10. IPA refers to isopropyl alcohol, EDTA refers to ethylyene diaminetetraacetic acid, B3 refers to niacinamide, 12HSA refers to 12-hydroxystearic acid, DC2501 wax refers to DOWSIL™ 2501 a water dispersible silicone glycol copolymer wax, which has a low melting point. It has many benefits including moisturizing, tackiness reduction, and foam boosting. PEG400 refers to is a low-molecular-weight grade of polyethylene glycol. It is a clear, colorless, viscous liquid. PPG15 stearyl ether refers to a polypropylene glycol ether of stearyl ether. PPG-2 isoceteth-20 acetate refers to polypropylene glycol isoceteth acetate. IPM refers to isopropyl myristate.

TABLE 1
All numbers are shown as weight %

| | Sample # | | | | | | | | |
|---|---|---|---|---|---|---|---|---|---|
| | 1 | 2 | 3 | 4 | 5 | 6 | 7 | 8 | 9 |
| Ingredient | Quantity present (% by weight in composition) | | | | | | | | |
| Water | q.s | q.s | q.s | q.s. | q.s. | q.s. | q.s. | q.s. | q.s. |
| BENECEL ™ E10M (nonionic) | 1.5 | 1.5 | 1.5 | 1.5 | | | 1.5 | 1.5 | 1.5 |
| KLUCEL ™ (nonionic) | | | | | 1.1 | | | | |
| PQ-10 (cationic) | | | | | | 0.8 | | | |
| Alcohol (ethanol) | 62 | 62 | 62 | 62 | 62 | 62 | 62 | 62 | 62 |
| IPA | 3 | 3 | 3 | 3 | 3 | 3 | 3 | 3 | 3 |
| Tocopherol | 0.05 | 0.05 | 0.05 | 0.05 | 0.05 | 0.05 | 0.05 | 0.05 | 0.05 |
| EDTA | 0.005 | 0.005 | 0.005 | 0.005 | 0.005 | 0.005 | 0.005 | 0.005 | .005 |
| Fragrance | 0.2 | 0.2 | 0.2 | 0.2 | 0.2 | 0.2 | 0.2 | 0.2 | 0.2 |
| B3 | 1 | 1 | 1 | 1 | 1 | 1 | 1 | 1 | 1 |
| PDADMAC (40%) | 1.25 | 2.5 | 1.25 | 1.25 | 1.25 | 1.25 | 1.25 | 1.25 | 1.25 |
| 12HSA | 0.01 | 0.01 | 0.01 | 0.01 | 0.01 | 0.01 | 0.01 | 0.01 | 0.01 |
| Propylene glycol | | | 5 | | | | | | |
| PEG400 | 2 | 2 | | 2 | 2 | 2 | 2 | 2 | 2 |
| Diisopropyl adipate | 5 | 5 | 3 | | | 5 | | | |
| C12/C14/C16 lactate ester | | | | 5 | 5 | | | | |
| PPG15 stearyl ether | | | | | | | | 5 | |
| Diethyl hexyl malate | | | | | | | 5 | | |
| Diisopropyl adipate/IPM (3.5/1.5) | | | | | | | | | 5 |
| Transparent | Yes | Yes | Yes | Yes | Yes | Yes | Yes | Yes | Yes |
| Water solubility in oil | No | No | No | No | No | No | No | No | No |
| Initial Viscosity (Brookfield, | 4000 | 4200 | | 4000 | 6580 | 2140 | 3620 | 5080 | 4080 |
| spindle 4, 10 RPM, cps) | | | | | | | | | |

| | |
|---|---|
| Solubility of oil 75/25 EtOH/Water | >30% |
| Sensory | Non-tacky |

TABLE 2
All numbers are shown as weight %

| | Sample # | | | | | | | | | | |
|---|---|---|---|---|---|---|---|---|---|---|---|
| | CE1 | CE2 | CE3 | CE4 | CE5 | CE6 | CE7 | CE8 | CE9 | CE10 | CE11 |
| Ingredient | Quantity present (% by weight in composition) | | | | | | | | | | |
| Water | q.s. | q.s. | q.s | q.s. | q.s. | q.s. | q.s. | q.s. | q.s. | q.s. | q.s. |
| Benecel E10M | | 1.5 | 1.5 | 1.5 | 1.5 | 1.5 | 1.5 | 1.5 | 1.5 | 1.5 | 1.5 |
| Klucel | 1.5 | | | | | | | | | | |
| Alcohol (ethanol) | 62 | 62 | 62 | 62 | 62 | 62 | 62 | 62 | 62 | 62 | 62 |
| IPA | 3 | 3 | 3 | 3 | 3 | 3 | 3 | 3 | 3 | 3 | 3 |
| Tocopherol | 0.05 | 0.05 | 0.05 | 0.05 | 0.05 | 0.05 | 0.05 | 0.05 | 0.05 | 0.05 | 0.05 |
| EDTA | 0.005 | 0.005 | 0.005 | 0.005 | 0.005 | 0.005 | 0.005 | 0.005 | 0.005 | 0.005 | 0.005 |
| Fragrance | 0.2 | 0.2 | 0.2 | 0.2 | 0.2 | 0.2 | 0.2 | 0.2 | 0.2 | 0.2 | 0.2 |
| B3 | 1 | 1 | 1 | 1 | 1 | 1 | 1 | 1 | 1 | 1 | 1 |
| PDADMAC | 1.25 | 1.25 | 1.25 | 1.25 | 1.25 | 1.25 | 1.25 | 1.25 | 1.25 | 1.25 | 1.25 |
| (40%) | | | | | | | | | | | |
| 12HSA | 0.01 | 0.01 | 0.01 | 0.01 | 0.01 | 0.01 | 0.01 | 0.01 | 0.01 | 0.01 | 0.01 |
| PEG400 | 2 | 2 | 2 | 2 | 2 | 2 | 2 | 2 | 2 | 2 | 2 |
| PPG-2 Isoceteth- | | 5 | | | | | | | | | |
| 20 acetate | | | | | | | | | | | |
| Glycereth-5 | | | 5 | | | | | | | | |
| lactate | | | | | | | | | | | |
| Glycereth-7 | | | | 5 | | | | | | | |
| triacetate | | | | | | | | | | | |
| Dioctyl succinate | | | | | 5 | 1 | | | | | |
| Dicapryl maleate | | | | | | | 5 | | | | |
| Diethyl hexyl | | | | | | | | 5 | | | |
| sebacate | | | | | | | | | | | |
| Diethyl hexyl | | | | | | | | | 5 | | |
| adipate | | | | | | | | | | | |
| IPM | | | | | | | | | | 5 | |
| Transparent | Yes | Yes | Yes | Yes | No | No | No | No | No | No | No |
| Water solubility | — | Yes | Yes | Yes | No | No | No | No | No | No | No |
| in oil | | | | | | | | | | | |
| Initial Viscosity | 3840 | 3920 | 3640 | 4240 | 4060 | 3960 | 4580 | 3640 | 3720 | 4940 | 2720 |
| (Brookfield, | | | | | | | | | | | |
| spindle 4, | | | | | | | | | | | |
| 10 RPM, cps) | | | | | | | | | | | |

| | | | |
|---|---|---|---|
| Solubility | — | >30% | <10% |
| of oil 75/25 | | | |
| EtOH/Water | | | |

| | |
|---|---|
| Sensory | Tacky |

The level of alcohol present in the antimicrobial composition can be adjusted based upon the purity percentage of the alcohol. For example, the ethanol purity should preferably be 92% w/w to 94% w/w such that if using ethanol with a purity of 92%, the ethanol can be used in an amount of 70 to 80% by weight, for example, 72% by weight.

The compositions in Table 1 and Table 2 can be converted into a low viscosity spray formulation by simply removing the structurants Benecel E10m,Klucel or cationic structuring polymers. Table 4 shows detackifying agents that can be used in the antimicrobial compositions to design a clear hand sanitizer formulation with a non-tacky feel. The examples are not meant to limit the materials that can be used but merely to illustrate detackifying agents type and level selected based on three criteria. The detackifying agents that are preferred for use are detackifying agents that have greater than 30% solubility in at least a 75/25 EtOH/Water blend reflective of good solubility in the formulations in Table 1.

Furthermore, the detackifying agent must be liquid at room temperature so that once left behind as a film, the composition does not impart any negative (sticky/tacky) feel. Finally, the preferred detackifying agent must itself not have solvency for water such that during application and on drying as the ethanol and water evaporate, the water is also allowed to quickly evaporate leaving behind only the oil and any skin benefit actives/moisturizers if present. Without wishing to be bound by theory it is believed that when a hand sanitizer formulation with preferred detackifying agents as disclosed herein is applied to the skin and as the water and ethanol evaporate, if the detackifying agent possesses good solubilizing capacity for water, this will delay evaporation and drive towards an undesirable slower drying and tackier feel.

In the formulations listed in Table 2, the detackifying agents listed fail in one or more of these criteria and thus, are outside the scope of the presently disclosed antimicrobial compositions.

Sensory evaluation of select inventive and comparative compositions was done. Samples 1 and 4 were assessed versus a control formulation containing no sensory oil, CE1, and two other comparative compositions, CE3 and CE4. Small sample jars of each of the five compositions were given to seven individuals who were then asked to rank order samples in regard to tackiness. Sensory assessment results indicate that comparative examples CE1, CE2 and CE3 all had significantly more tack than Samples 1 and 4, containing the inventive samples disclosed herein. Such a result indicates that the antimicrobial compositions disclosed herein containing a detackifying agent provide a non-tacky feeling to the user.

### Example 2

Samples 10-14 are further examples demonstrating compositions of the antimicrobial compositions disclosed herein. Samples 10-14 are made with the same process described with respect to Samples 1-9.

TABLE 3
All numbers are shown as weight %

| | Sample # | | | | |
|---|---|---|---|---|---|
| | 10 | 11 | 12 | 13 | 14 |
| Ingredient | Quantity present (% by weight in composition) | | | | |
| Water | q.s | q.s | q.s | q.s. | q.s. |
| SoftCAT ™ SK-MH | 2 | | | | |
| Soft CAT ™ SX-1300X | | 2 | | | |
| Soft CAT ™ SX-1300H | | | 2 | | |
| UCARE ™ KG-30M | | | | 2 | |
| UCARE ™M JR-30M | | | | | 2 |
| Alcohol (ethanol) | 62 | 62 | 62 | 62 | 62 |
| IPA | 3 | 3 | 3 | 3 | 3 |
| Tocopherol | 0.05 | 0.05 | 0.05 | 0.05 | 0.05 |
| EDTA | 0.005 | 0.005 | 0.005 | 0.005 | 0.005 |
| Fragrance | 0.2 | 0.2 | 0.2 | 0.2 | 0.2 |
| B3 | 1 | 1 | 1 | 1 | 1 |
| PDADMAC (40%) | 1.25 | 2.5 | 1.25 | 1.25 | 1.25 |
| 12HSA | 0.01 | 0.01 | 0.01 | 0.01 | 0.01 |
| Propylene glycol | | | 5 | | |
| PEG400 | 2 | 2 | | 2 | 2 |
| Diisopropyl adipate | 5 | | 3 | | |
| C12/C14/C16 lactate ester | | 5 | | | |
| PPG15 stearyl ether | | | | 3 | |
| Diethyl hexyl malate | | | | | 5 |
| Transparent | Yes | Yes | Yes | Yes | Yes |
| Water solubility in oil | No | No | No | No | No |

| | |
|---|---|
| Solubility of oil 75/25 EtOH/Water | >30% |
| Sensory | Non-tacky |

Listed in Table 4 are examples of detackifying agents that satisfy and do not satisfy the three criteria, which include: (1) the oil must be liquid; (2) the oil must be soluble in at 7 least a 75/25 ethanol/water mixture; and (3) the oil must have no solvency for water and thus, is desirable for use in the disclosed antimicrobial compositions. RT in Table 4 refers to room temperature.

TABLE 4

| | Solubility in | Water | Oil | |
|---|---|---|---|---|
| | 75/25 | solubility in | (liquid or | |
| | EtOH/ | detackifying | solid at | In or out |
| Detackifying agent | Water | agent | RT) | of scope |
| Dicapryl maleate | <10% | No | Liquid | Out |
| Dioctyl succinate | <10% | No | Liquid | Out |
| Diethyl hexyl sebacate | <10% | No | Liquid | Out |
| Diethylhexyl adipate | <10% | No | Liquid | Out |
| PPG-2-Isoceteth-2 | >30% | Yes | Liquid | Out |
| PPG15 Stearyl ether | >30% | No | Liquid | In |
| Glycereth-5 lactate | >30% | Yes | Liquid | Out |
| Glycereth-7 triacetate | >30% | Yes | Liquid | Out |
| Disopropyl adipate | >30% | No | Liquid | In |
| Diisopropyl sebacate | >30% | No | Liquid | In |
| Lauryl lactate | >30% | No | Liquid | In |
| CCT | <10% | No | Liquid | Out |
| Lauryl lactate/IPM (80/20) | >30% | No | Liquid | In |
| Lauryl lactate/IPM (5/50) | <10% | No | Liquid | Out |
| Isostearyl alcohol | >30% | No | Liquid | In |
| Diisopropyl adipate/IPM | >30% | No | Liquid | In |
| 80/20 | | | | |
| Diethyl hexyl malate | >30% | No | Liquid | In |
| PPG-2 Isoceteth-20 | >30% | no | Solid | Out |
| Acetate | | | | |

1. An antimicrobial composition, comprising:
water;
60 to 70% by weight of ethanol;
0.5 to 3.0 wt % of a cationic polymer being poly diallyl dimethyl ammonium chloride; and
0.5 to 10 wt % of a detackifying agent, wherein the detackifying agent comprises a soluble detackifying agent selected from diisopropyl adipate, diisopropyl sebacate, or diethyl hexyl malate, or a combination thereof; and
a skin benefit agent, wherein the skin benefit agent comprises niacinamide, tocopherol, aloe vera, sodium or potassium hyaluronate, alpha-hydroxy acids and esters, hydroxy stearic acid, beta-hydroxy acids and esters, hydroxyethyl urea, polyhydroxy acids and esters, creatine, hydroquinone, t-butyl hydroquinone, mulberry extract, liquorice extract, or a combination thereof.

2. The antimicrobial composition of claim 1, wherein the cationic polymer has a percent nitrogen content of greater than or equal to 5% by weight of the cationic polymer.

3. (canceled)

4. (canceled)

5. The antimicrobial composition of claim 1, wherein the detackifying agent is liquid, wherein the detackifying agent is soluble in at least a 75/25 ethanol/water mixture, and wherein there is no water solubility in the detackifying agent.

6. (canceled)

7. The antimicrobial composition of claim 1, wherein the detackifying agent comprises combination of a soluble detackifying agent and an insoluble detackifying agent, wherein the insoluble detackifying agent is oil that has solubility in at least 75/25 or higher ratio of ethanol/water of less than 10%, wherein the combined detackifying agent is liquid at room temperature, is soluble in at least a 75/25 ethanol/water mixture, and wherein there is no water solubility in the detackifying agent.

8. The antimicrobial composition of claim 1, further comprising a humectant, wherein the humectant comprises glycerin, butylene glycol, propylene glycol, polyethylene glycols, sorbitol, polyglycerol, isoprene glycol, hyaluronic acid, or a combination thereof.

9. (canceled)

10. The antimicrobial composition of claim 1, wherein the antimicrobial composition is in the form a liquid, gel, or spray.

11. The antimicrobial composition of claim 1, further comprising a viscosity adjusting structurant, wherein the structurant is selected from a nonionic structurant, a cationic structurant, or a combination thereof.

12. The antimicrobial composition of claim 1, wherein the antimicrobial composition is transparent and wherein addition of the detackifying agent does not affect the transparency of the antimicrobial composition.
